(19) United States
(12) Patent Application Publication
(10) Pub. No.: US 2024/0325334 A1
(43) Pub. Date: Oct. 3, 2024

(54) COMPOSITION FOR GENERATING NITROGEN MONOXIDE

(71) Applicant: SEOUL NATIONAL UNIVERSITY R&DB FOUNDATION, Seoul (KR)

(72) Inventors: YOUNG BIN CHOY, Seoul (KR); HAN BI JI, Seoul (KR); SE NA KIM, Chungcheongbuk-do (KR)

(21) Appl. No.: 18/681,600

(22) PCT Filed: Sep. 6, 2022
(86) PCT No.: PCT/KR2022/013316

§ 371 Date: Feb. 6, 2024

(30) Foreign Application Priority Data
Sep. 13, 2021 (KR) 10-2021-0121523
Sep. 6, 2022 (KR) 10-2022-0112531

(51) Int. Cl. A61K 31/198; A61K 31/4745; A61K 31/555; A61K 33/24; A61K 33/26; A61K 33/30; A61K 33/32; A61K 33/34; A61P 35/00

(52) U.S. Cl. CPC A61K 31/198; A61K 31/4745; A61K 31/555; A61K 33/24; A61K 33/26; A61K 33/30; A61K 33/32; A61K 33/34; A61P 35/00

(57) ABSTRACT

A composition for generating nitric oxide includes a material containing a transition metal, and a compound. The composition for generating nitric oxide of the present invention may increase the level of nitric oxide to inhibit anticancer drug resistance and improve effects of anticancer chemotherapy.

### CROSS REFERENCE TO RELATED APPLICATIONS AND CLAIM OF PRIORITY

This application claims benefit under 35 U. S. C. 119, 120, 121, or 365 (c), and is a National Stage entry from International Application No. PCT/KR2022/013316, filed Sep. 6, 2022, which claims priority to the benefit of Korean patent application Nos. 10-2021-0121523 filed on Sep. 13, 2021, and 10-2022-0112531 filed on Sep. 6, 2022, in the Korean intellectual property office, the entire contents of which are incorporated herein by reference.

### BACKGROUND

### 1. Technical Field

The present invention relates to a composition for generating nitrogen monoxide.

### 2. Background Art

Nitrogen monoxide (or nitric oxide; NO) is generated in various cells of the human body, and is known as a defense substance that exhibits anticancer and antimicrobial actions in the immune system, a neurotransmitter in the nervous system, and a vasodilator in the circulatory system.

In order to use nitric oxide, there is a method of directly supplying nitric oxide to a breathing tube by including nitric oxide in breathing gases as a type of nitric oxide delivery method, but there are problems in that the half-life of nitric oxide is short and high concentration of nitric oxide is toxic.

Accordingly, a method of producing nitric oxide by supporting nitric oxide precursors such as S-nitrosothiol, diazeniumdiolates, and NO-metal composite on a carrier has been studied. However, there is a problem in that most of them are decomposed, and then may be converted into carcinogenic or inflammatory by-products.

Thus, the need for production of non-toxic and biocompatible nitric oxide has emerged. The present inventors have completed a composition for generating nitric oxide, which produces nitric oxide without toxicity using a compound that generates nitric oxide in an environment in which H₂O₂ exists along with a transition metal that catalyzes the same.

### SUMMARY

An object of the present invention is to provide a composition for generating nitric oxide, which may generate nitric oxide in a biocompatible manner while minimizing a problem of toxicity.

Another object of the present invention is to provide a pharmaceutical composition for anticancer effects or for inhibiting anticancer drug resistance, which includes the composition for generating nitric oxide.

In addition, another object of the present invention is to provide a pharmaceutical composition for preventing or treating cardiovascular disease or infertility, or for treating wounds, which includes the composition for generating nitric oxide.

1. A composition for generating nitric oxide, including: a material containing iron; and L-arginine.

2. A composition for generating nitric oxide, including: a material containing a transition metal; and a compound represented by Formula 1 below or a salt thereof:

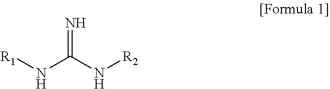

wherein, in the above formula, R₁ is hydrogen or an alkyl group having 1 to 5 carbon atoms, and R₂ is a substituted or unsubstituted alkyl group having 1 to 30 carbon atoms.

3. The composition according to the above 2, wherein R₂ is substituted with a carboxyl group or an ester group having 1 to 10 carbon atoms.

4. The composition according to the above 2, wherein the material is a carrier, and the compound is supported on the carrier.

5. The composition according to the above 2, wherein the transition metal is one selected from the group consisting of iron, manganese, zirconium, copper, nickel, cobalt and zinc.

6. The composition according to the above 4, wherein the carrier is a metal-organic framework.

7. The composition according to the above 6, wherein the metal-organic framework includes a porphyrin ligand.

8. A pharmaceutical composition for preventing or treating cancer, including the composition for generating nitric oxide according to any one of the above 1 to 7.

9. A pharmaceutical composition for inhibiting anticancer drug resistance, including the composition for generating nitric oxide according to any one of the above 1 to 7.

10. The pharmaceutical composition according to the above 9, wherein the anticancer drug is Irinotecan, Eribulin, Carboplatin, Cisplatin, Halaven, 5-fluorouracil (5-FU), Gleevec, Vincristine, Vinblastine, Vinorelvine, Paclitaxel, Docetaxel, Etoposide, Topotecan, Dactinomycin, Doxorubicin, Daunorubicin, Valrubicin, Flutamide, Gemcitabine, Mitomycin, Bleomycin, Capecitabine, Tamoxifen, Belotecan, Imatinib, Streptozocin, Mechlorethamine, Chlorambucil, Doxifluridine, Etoposide, Idarubicin, Letrozole, Leucovorin, Levamisole, Teniposide, Tretinoin, Sorafenib, Mitoxantrone, Thioguanine, Pipobroman, Mesna, Nofetumomab, Oprelvekin, Cytarabine, Dacarbazine or Afinitor.

11. A pharmaceutical composition for preventing or treating hypertension or arteriosclerosis, including the composition for generating nitric oxide according to any one of the above 1 to 7.

12. A pharmaceutical composition for treating wounds, including the composition for generating nitric oxide according to any one of the above 1 to 7.

13. A pharmaceutical composition for preventing or treating infertility, including the composition for generating nitric oxide according to any one of the above 1 to 7.

The present invention may inhibit anticancer drug resistance by generating nitric oxide in a non-toxic and biocompatible manner thus to improve effects of anticancer chemotherapy, and may be used to prevent or treat cancer, cardiovascular disease, genital disease, or to treat wounds.

### BRIEF DESCRIPTION OF THE DRAWINGS

FIG. 1 is a SEM image of PCN-223-Fe prepared according to an embodiment of the present invention, which shows the shape and size of the particles.

FIG. 2 shows XRD analysis results of PCN-223-Fe prepared according to an embodiment of the present invention.

FIG. 3 shows BET analysis results of PCN-223-Fe prepared according to an embodiment of the present invention.

FIG. 4 shows L-arginine loading amount and in-vitro release profile in L-arginine-loaded particles (L-arg@PCN-223-Fe).

FIG. 5 shows irinotecan loading amount and in-vitro release profile in irinotecan-loaded particles (IRI@PCF-223-Fe).

FIG. 6 shows amounts of NO generated in a mixed formulation of L-arg@PCN-223-Fe and IRI@PCN-223-Fe according to the concentration of H₂O₂.

FIG. 7 shows results of comparing the degree of intracellular NO generation by treatment group (Ctrl: no treatment group, L-arg solution: a group treated with L-arginine solution, PCN-223-Fe: a group treated with PCN-223-Fe, L-arg@PCN-223-Fe: a group treated with L-arginine loading particles).

FIG. 8 shows cell viability by treatment group for assessment of antitumor efficacy at the cell level (IRI sol: a group treated with irinotecan, IRI sol+L-arg@PCN-223-Fe: a group treated with irinotecan solution and L-arginine loading particles, IRI@PCN-223-Fe: a group treated with irinotecan loading particles, IRI@PCN-223-Fe+L-arg@PCN-223-Fe: a group treated with irinotecan loading particles and L-arginine loading particles).

### DETAILED DESCRIPTION

Hereinafter, the present invention will be described in detail.

The present invention relates to a composition for generating nitric oxide.

The composition of the present invention may include: a material containing iron; and L-arginine.

The composition of the present invention may include: a material containing a transition metal; and a compound represented by Formula 1 below or a salt thereof.

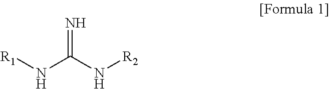

Wherein, in the above formula, R₁ may be hydrogen or an alkyl group having 1 to 5 carbon atoms, and R₂ may be a substituted or unsubstituted alkyl group having 1 to 30 carbon atoms.

In the present specification, the “alkyl group” means a straight-chain or branched saturated hydrocarbon group. The alkyl group may be substituted or unsubstituted.

The alkyl group may include, for example, methyl, ethyl, n-propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 1-methylhexyl, 2-methylhexyl, 1-ethylpentyl, 2-ethylpentyl, 1-propylbutyl, 1-ethyl-2-methylpropyl, n-octyl, n-nonyl, n-decyl, isodecyl, 2-propylheptyl and the like.

As used herein, “substitution” means that one or more hydrogen atoms in a structure are replaced with the same or different substituents, independently of each other.

Herein, the substituent may include, for example, halogen, azide, alkyl, aralkyl, alkenyl, alkynyl, cycloalkyl, hydroxyl, alkoxyl, amino, nitro, imino, amido, carbonyl, carboxyl, nitrile, silyl, ether, alkylthio, sulfonamino, sulfhydryl, sulfide, disulfide, sulfinyl, sulfonyl, sulfino, sulfo, carbonothioyl, thioester, thionoester, hydroxy, alkoxy, ketone, aldehyde, ester, heterocyclyl, aromatics or heteroaromatics, aryl, allyloxy, heteroaryl, arylalkyl, acylalkyl, carboxyester, aminoalkyl, alkylaminoaryl, alkylaryl, alkylaminoalkyl, alkoxyaryl, arylamino, aralkylamino, alkylsulfonyl, haloalkyl, etc., and one or more of them may be linked or substituted with each other, but they are not limited thereto. The position of the substituent is not particularly limited.

For example, the alkyl group of R₂ may be substituted with a carboxyl group or an ester group having 1 to 10 carbon atoms.

For example, R₂ may be in the form of

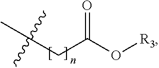

wherein n may be an integer from 1 to 10, and R₃ may be hydrogen or a substituted or unsubstituted alkyl group having 1 to 10 or 1 to 5 carbon atoms.

For example, R₂ may be in the form of

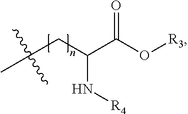

wherein n may be an integer from 1 to 10, R₃ may be hydrogen or a substituted or unsubstituted alkyl group having 1 to 10, or 1 to 5 carbon atoms, and R₄ may be hydrogen or a substituted or unsubstituted alkyl group having 1 to 10, or 1 to 5 carbon atoms.

R₂ may be a substituted or unsubstituted alkyl group having 1 to 20, or 1 to 10 carbon atoms.

The compound may be, for example, L-arginine, or an analogue of L-arginine.

“L-arginine” is a type of amino acid, and is a precursor of nitric oxide (NO), which reacts with H₂O₂ to generate nitric oxide and non-toxic by-products.

The “analog” that means some of the atoms constituting the organic compound molecule are replaced by other types of elements to have a structure corresponding to the original compound and to exhibit functions similar to those of the substance.

Specifically, the L-arginine analog includes a functional group

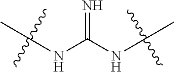 [SMILES: *NC(=N)N*]

of L-arginine, such that L-arginine may be substituted with a corresponding structure while maintaining the function of generating nitric oxide in an environment in which hydrogen peroxide exists.

For example, the above compound may be one selected from the group consisting of the following compounds, but it is not limited thereto.

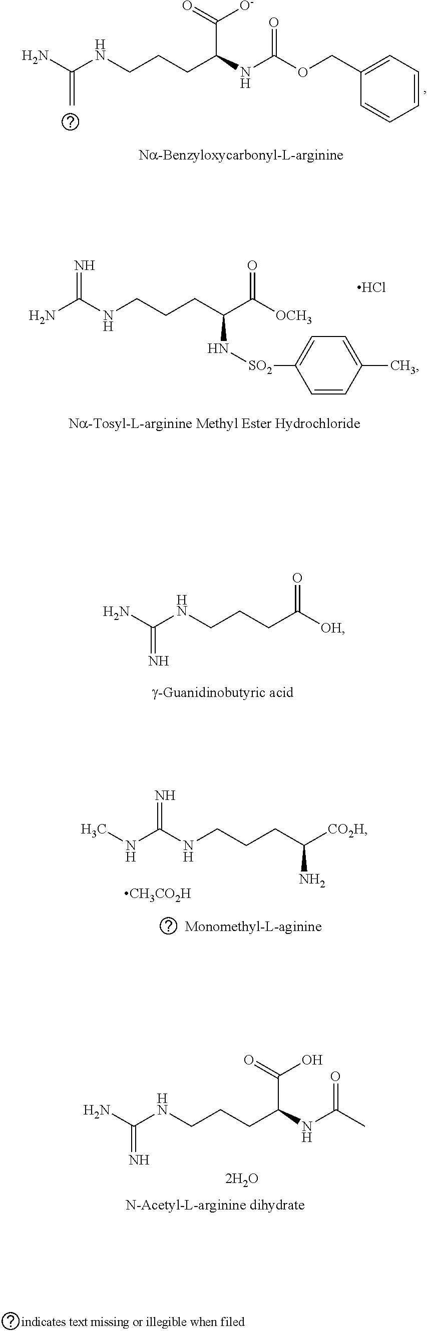

As used herein, the salt means a salt prepared using a specific compound according to the present invention and a relatively non-toxic acid or base. The salt may be, for example, an acid addition salt or metal salt. The salt may be a pharmaceutically acceptable salt.

The transition metal is not particularly limited to elements belonging to groups 3 to 12 in the periodic table, and may be, for example, one selected from iron, manganese, zirconium, copper, nickel, cobalt and zinc, and preferably iron or manganese. The transition metal may include one or two or more types of transition metals.

In the composition of the present invention, since the transition metal serves as a catalyst when generating nitric oxide from the compound, the material is not particularly limited as long as it includes a transition metal.

For example, the material may be a molecule, nanoparticle or transition metal complex containing a transition metal, and the shape, raw material or size thereof is not limited. In the case of the shape, for example, particle, sheet or any other form may be used. When the material is in the form of particles, the particle may have a size of 50 nm to 1000 μm, for example, but it is not limited thereto. Further, materials having different particle sizes may be used depending on the purpose and use.

In the case of the raw material, it may be, for example, metal, organic material, inorganic material, or a mixture thereof.

The metal raw material may be iron, aluminum, yttrium, titanium, zirconium, vanadium, niobium, chromium, molybdenum, tungsten, manganese, cobalt, nickel, copper, gold, platinum, silver or zinc, but it is not limited thereto.

The organic raw material may include, for example, polyethylene, polypropylene, polystyrene, polyester, polyimide, polyacrylic acid, polymethacrylic acid, polyolefin, polyurethane, agarose, dextran, dendrimer, hyaluronic acid, chitosan, albumin, gelatin, pectin, liposome, graphite, Vulcan X, Denka Black or Ketjen Black, but it is not limited thereto.

The inorganic raw material may be glass, silicon, silicon oxide, mullite, bauxite, celite, zeolite, calcium carbonate, silica, zirconia, titania, alumina or boehmite, but it is not limited thereto.

For example, when the transition metal is iron, the material may be nanoparticles such as FeO, Fe₃O₄, FePt, α-Fe₂O₃ or MnFe₂O₄; iron(II) chloride tetrahydrate, iron(II) acetate, bis(pentamethyl cyclopentadienyl) iron(II), ferric citrate, diferric dicitrate, triferric tricitrate, etc.; or iron-coordinated phthalocyanine based iron(II) phthalocyanine, iron(III) phthalocyanine-4,4′,4″,4′″-tetrasulfonic acid or porphyryn-based Fe-5,10,15,20-tetra-pyridyl-porphyrin.

The transition metal may be supported on the material, bonded to an outside or inside of the material, or included in a framework constituting the material.

“Supporting” means a state in which any component is included in the material through a physical or chemical bonding.

“Bonding” may be performed through ionic bonding, covalent bonding, coordination bonding, hydrogen bonding, electrostatic attraction, van der Waals force, hydrophobic bonding, etc., but it is not limited thereto.

A substance having a functional group may be used for being supported or binding to the above material, and it may be supported or bonded to the material or included in the framework of the material through a reaction between the above functional group and a functional group on the surface of the material. As long as the functional group can form a bond through a coupling reaction, functional groups known in the art may be used without limitation. For example, it may be a hydroxyl, carboxyl, carbonyl, amino, thiol, phosphoric acid, hydroxyphenyl, or carboxyphenyl group.

As a specific example, when the transition metal is iron, Fe-THPP (Fe-tetra(p-hydroxyphenyl) porphyrin), Fe-m-THPP (Fe-tetra(m-hydroxyphenyl) porphyrin) or Fe-TCPP (Fe-tetra(4-carboxyphenyl) porphyrin) may be reacted with the material, thus to be supported or bonded to the material, or otherwise, may be included in a reaction during formation of the material so that iron may be included in the framework constituting the material.

The material may include, for example, a transition metal and a ligand coordinated thereto.

“Ligand” is also referred to as a coordination ligand, and is a generic term for ions or molecules which are bonded to the periphery of a core metal ion of the coordinated compound.

For example, a substance known as a heme ligand or a non-heme ligand used in an oxygenase containing a metal at an active site may be used as the ligand. The metal is not particularly limited as long as it is a transition metal.

The heme ligand refers to a porphyrin ligand that binds to a core metal like a heme structure in the body.

“Porphyrin” is a generic term for macrocyclic compounds in which four pyrrole units are linked by a methine group (═CH—).

A type of metal porphyrin in which divalent or trivalent iron ions are coordinated to porphyrin may include, for example,

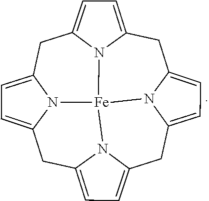

As the above core metal, iron may be replaced with another metal corresponding to any other transition metal.

The non-heme ligand may be, for example, one selected from the group consisting of the following compounds, but it is not limited thereto.

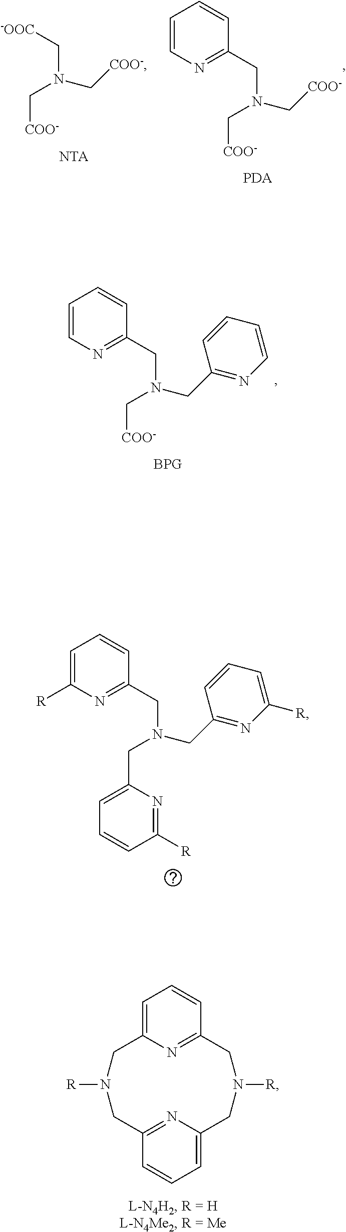

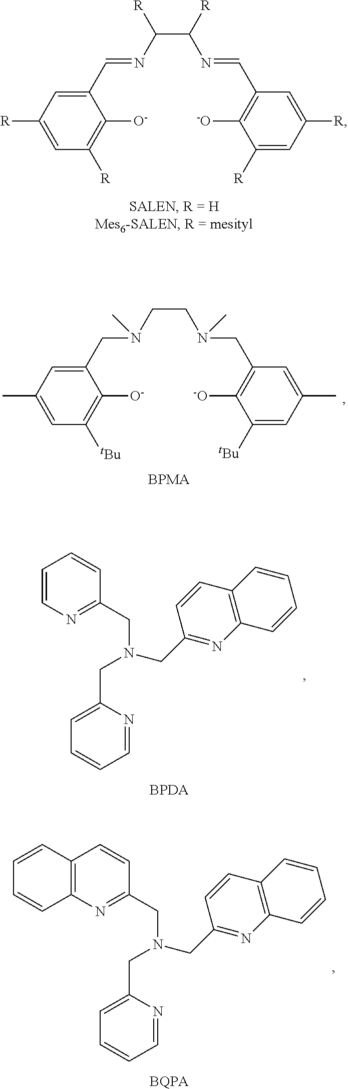

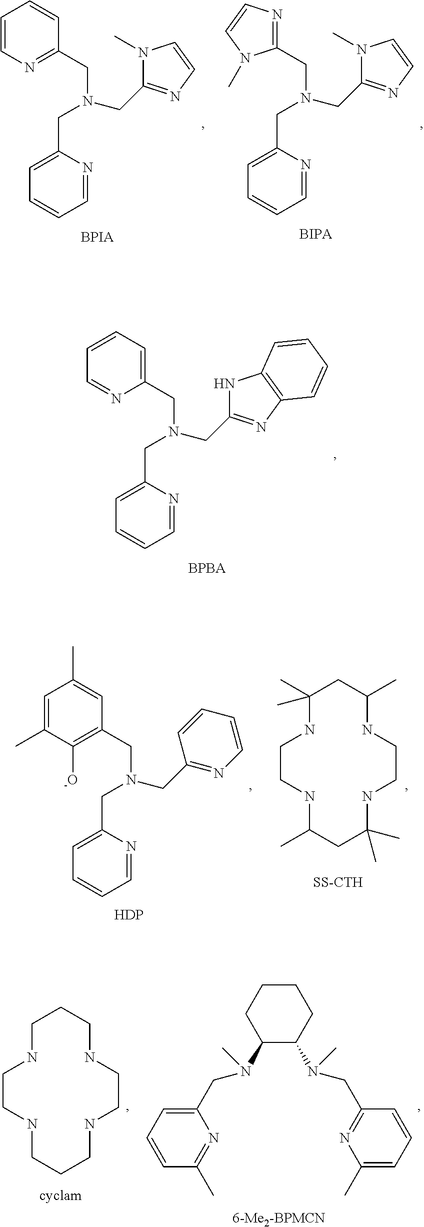

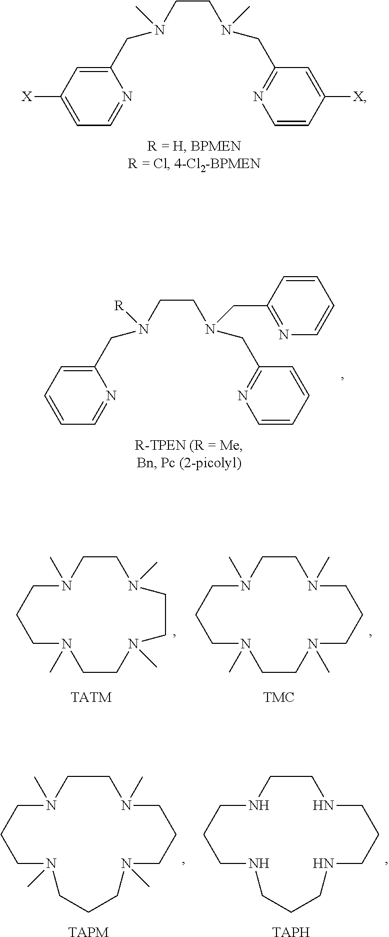

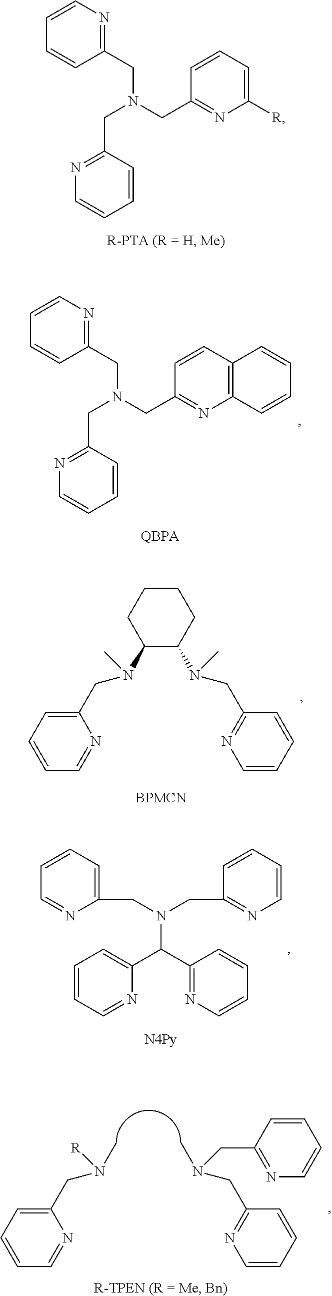

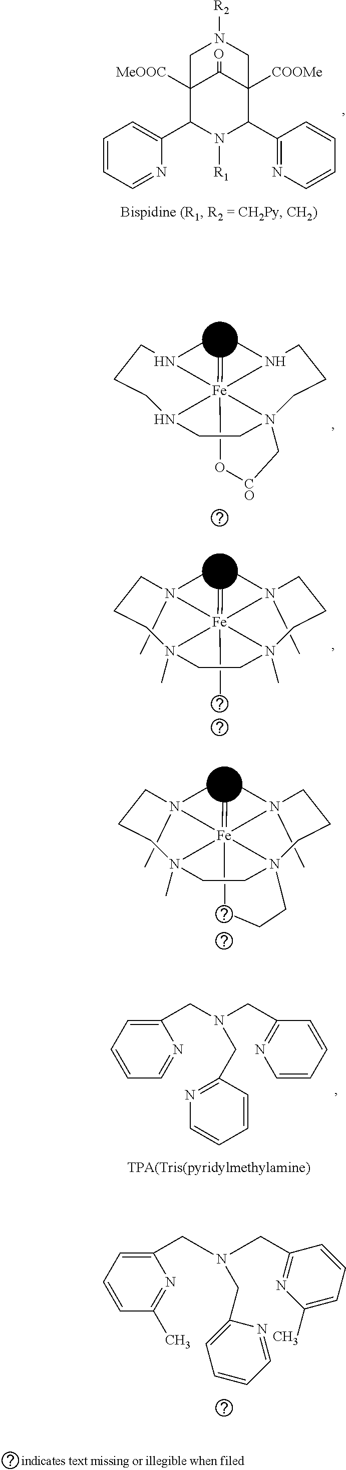

Iron in the above formula may be replaced with other transition metals.

The material may be a carrier, and the compound may be supported on the inside or outside of the carrier. This for example, by various types of may be performed, attraction, bonding, etc. exemplified above.

The material may be, for example, a metal-organic framework (MOF).

The metal-organic framework, as a type of the carrier, refers to a porous crystal structure formed by infinitely linking three-dimensional metal ions or metal ion clusters as a result of self-assembly of organic ligands through coordination or covalent bonding, and may have a large specific surface area and high porosity.

Details of the ligand as described above.

The composition of the present invention may produce nitric oxide with high stability and without toxicity by supporting a compound that serves as a substrate for generating nitric oxide in an environment in which hydrogen peroxide exists on a metal-organic framework carrier which includes a transition metal, and then, delivering the compound into the living body.

The metal-organic framework may include, for example, a ligand coordinated to a transition metal as a component constituting the framework thereof.

For example, PCN-223 may be used as the metal-organic framework including the porphyrin.

A metal-organic framework including a porphyrin ligand coordinated to a transition metal as a configuration to form a framework thereof may be produced, for example, by mixing/reacting a compound, in which a functional group for a coupling reaction is bonded to the porphyrin ligand coordinated to the transition metal, with a precursor of the metal-organic framework. The precursor may be, for example, ZrCl₄, TiCl₄, CoCl₂, NiCl₂, MnCl₂, AgCl₂, FeCl₄, Mg(NO₃)₂, Mg(CH₃COO)₂, Cu(CH₃COO)₂, Zn(NO₃)₂ or Mn(NO₃)₂, but it is not limited thereto.

In addition, the present invention relates to a pharmaceutical composition for preventing or treating cancer, which includes the composition for generating nitric oxide.

Details of the composition for generating nitric oxide are the same as described above.

The composition for generating nitric oxide of the present invention may generate nitric oxide in an environment in which H₂O₂ exists.

Since H₂O₂ exists in excess amount in a cancer environment, the composition of the present invention may generate nitric oxide in the cancer environment, whereby cancer cells become extinct. Therefore, the composition of the present invention may be used for the purpose of preventing or treating cancer.

These cancers may include, for example, oral cancer, breast cancer, lung cancer, non-small cell lung cancer, stomach cancer, colon cancer, bone cancer, pancreatic cancer, skin cancer, head cancer, cervical cancer, skin cancer, malignant melanoma, uterine cancer, ovarian cancer, colon cancer, small intestine cancer, rectal cancer, perianal cancer, fallopian tube carcinoma, endometrial carcinoma, cervical carcinoma, vaginal carcinoma, vulvar carcinoma, esophageal cancer, small intestine tumor, lymph gland cancer, bladder cancer, gallbladder cancer, endocrine cancer, thyroid cancer, parathyroid cancer, adrenal cancer, soft tissue sarcoma, urethral cancer, penile cancer, prostate cancer, chronic leukemia, acute leukemia, lymphocytic lymphoma, bladder pressure, kidney cancer, ureteral cancer, renal cell carcinoma, renal pelvic carcinoma, spinal cord tumor, brainstem glioma or pituitary adenoma, but it is not limited thereto.

The composition of the present invention may support, for example, H₂O₂ or be administered together with H₂O₂ so as to produce a sufficient amount of nitric oxide.

The composition of the present invention may be administered in combination with an anticancer drug to increase the anticancer efficacy of the anticancer drug. In this case, the cancer may be an anticancer drug-resistant cancer.

With regard to the composition for generating nitric oxide, the nitric oxide generated in cancer cells may suppress the function of a drug efflux pump thus to prevent drug efflux in the cancer cells, and thereby maintaining the desired concentration of effective drugs in cells and eventually inhibiting anticancer drug resistance or multidrug resistance. Therefore, it is possible to maximize the anticancer efficacy of the anticancer drug administered in combination.

When the composition of the present invention is administered in combination with an anticancer drug, it may be administered before, simultaneously with, or after administration of the anticancer drug.

Further, the present invention relates to a pharmaceutical composition for inhibiting anticancer drug resistance, which includes the composition for generating nitric oxide, and may be used for inhibiting the resistance of a subject patient to other anticancer drugs.

Details of the composition for generating nitric oxide are the same as described above.

The cancer is a treatment target using the composition of the present invention, and details are the same as described above.

“Anticancer drug resistance” means that cancer cells cause genetic alterations in response to anticancer drugs, thereby avoiding the attack of anticancer drugs and weakening the effects of anticancer treatment. When cancer cells acquire resistance to a specific anticancer drug, they may have “multidrug resistance (MDR)”, a cross-resistance expressing resistance not only to the anticancer drug but also to other anticancer drugs having different structures.

The composition of the present invention may react with H₂O₂ present in cancer cells to generate nitric oxide, which inhibits anticancer drug resistance factors to prevent a drug efflux pump such as ATP-dependent transporters from functioning. Anticancer drug resistance or multidrug resistance may be inhibited by blocking the outflow of drugs into cancer cells to enable the concentration of effective drugs in cells to be maintained.

“Anticancer agent” refers to a therapeutic agent used in the treatment of hyperproliferative diseases such as cancer, and includes chemotherapeutic agents.

For example, the anticancer drug may be Irinotecan, Eribulin, Carboplatin, Cisplatin, Halaven, 5-fluorouracil (5-FU), Gleevec, Vincristine, Vinblastine, Vinorelvine, Paclitaxel, Docetaxel, Etoposide, Topotecan, Dactinomycin, Doxorubicin, Daunorubicin, Valrubicin, Flutamide, Gemcitabine, Mitomycin, Bleomycin, Capecitabine, Tamoxifen, Belotecan, Imatinib, Streptozocin, Mechlorethamine, Chlorambucil, Doxifluridine, Etoposide, Idarubicin, Letrozole, Leucovorin, Levamisole, Teniposide, Tretinoin, Sorafenib, Mitoxantrone, Thioguanine, Pipobroman, Mesna, Nofetumomab, Oprelvekin, Cytarabine, Dacarbazine or Afinitor, but it is not limited thereto.

Further, the composition of the present invention may be a pharmaceutical composition for preventing or treating hypertension or arteriosclerosis, which includes the composition for generating nitric oxide.

Details of the composition for generating nitric oxide are the same as described above.

“Nitric oxide (NO)” is a representative gas signaling substance involved in vasodilation, immune response regulation, anticancer and the like. As a vasodilator, nitric oxide is known to relax blood vessels, decrease blood pressure, and enhance blood flow, thereby preventing or treating cardiovascular diseases such as hypertension, angina pectoris, arteriosclerosis, and vasoconstrictive diseases.

The nitric oxide product of the present invention may increase a level of nitric oxide in the body such as in vascular endothelial cells, and may be used to prevent or treat cardiovascular diseases such as hypertension or atherosclerosis.

The composition of the present invention may support, for example, H₂O₂ or be administered together with H₂O₂ to produce a sufficient amount of nitric oxide.

Further, the composition of the present invention may be a pharmaceutical composition for treating wounds, which includes the composition for generating nitric oxide.

Details of the composition for generating nitric oxide are the same as described above.

“Nitric oxide (NO)” is a representative gas signaling substance involved in angiogenesis. Angiogenesis, the process of forming new microvessels, is an important component of wound healing, and nitric oxide is known to heal wounds by increasing angiogenesis in wounds. Further, nitric oxide is an essential substance for activation of angiogenesis-promoting cytokines, and is involved in activating vascular endothelial growth factor (VEGF), an angiogenic factor, in the form of angiogenesis.

The nitric oxide products of the present invention may effectively heal wounds by increasing the level of nitric oxide at the wound site.

The composition of the present invention may support, for example, H₂O₂ or be administered together with H₂O₂ to produce a sufficient amount of nitric oxide.

Further, the composition of the present invention may be a pharmaceutical composition for preventing or treating infertility, which includes the composition for generating nitric oxide.

Details of the composition for generating nitric oxide are the same as described above.

“Nitric oxide (NO)” is a signaling substance that transmits commands between cells and tissues, and is synthesized and/or secreted from the head of sperm, thereby causing various fertilization reactions within the egg. Nitric oxide is known to prevent or treat infertility by activating sperm or ovaries and participating in fertilized egg implantation and placenta formation. For example, a fertilizing ability may be increased by injecting nitric oxide into sperm insufficient with nitric oxide concentration.

The nitric oxide product of the present invention may increase the level of nitric oxide in the body, such as in the genitals or germ cells, and may be used to prevent or treat genital disorders such as infertility.

The composition of the present invention may support, for example, H₂O₂ or be administered together with H₂O₂ to produce a sufficient amount of nitric oxide. Hereinafter, the present invention will be described in detail through examples.

### EXAMPLE

### 1. Synthesis and Characteristic Analysis of PCN-223-Fe

### 1.1. Synthesis Method of PCN-223-Fe

First, solution A was prepared by dissolving 100 mg of Fe(III) meso-tetra(4-carboxyphenyl) porphine chloride and 2500 mg of benzoic acid in 10 ml of dimethylformamide (DMF), and 100 mg of zirconium chloride (ZrCl₄) was dissolved in 10 ml of DMF to prepare Solution B.

Then, solution A and solution B were mixed and stirred at room temperature for 30 minutes to prepare a mixture.

After stirring, the mixture of solution A and solution B was put into a 100 ml Duran bottle and reacted at 120° C. for 48 hours.

After completion of the reaction, washing was performed three times with DMF and methanol, respectively, followed by drying in a vacuum oven at 150° C. for 12 hours to remove the solvent, such that a metal-organic framework containing an iron-coordinated porphyrin ligand (hereinafter referred to as PCN-223-Fe) was obtained.

### 1.2. Characteristic Analysis of PCN-223-Fe

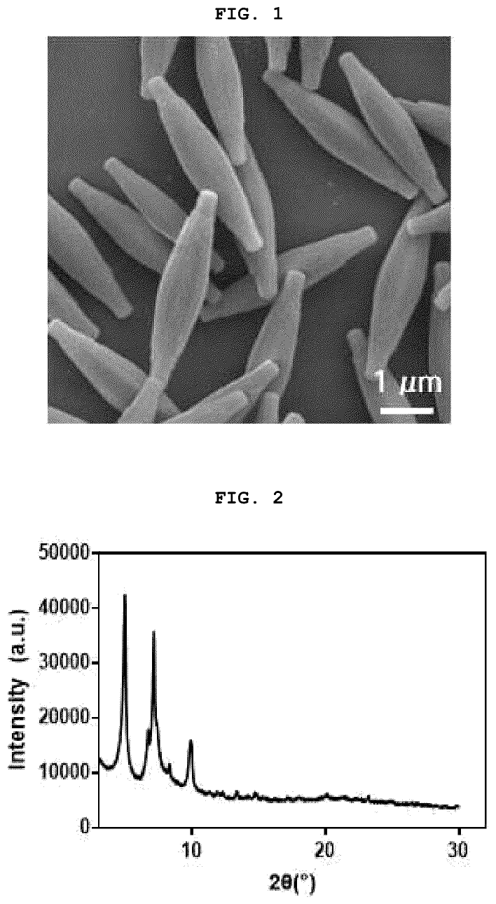

In order to determine the shape and size of the particles of PCN-223-Fe, the particles were observed by a scanning electron microscope (SEM), and results of the observation with SEM are shown in FIG. 1.

X-ray diffraction (XRD) was performed to analyze the crystal structure of PCN-223-Fe, and results thereof are shown in FIG. 2.

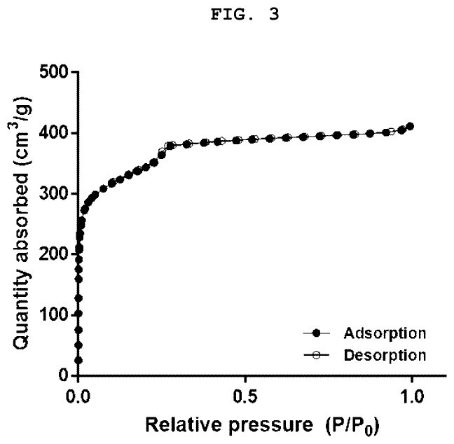

In order to confirm the specific surface area and pore size of PCN-223-Fe, N₂ adsorption-desorption test was performed, followed by analysis through BET (Brunauer, Emmett, Teller) calculation method, and results thereof are shown in FIG. 3.

### 2. L-Arginine (L-Arg) Loading and Analysis of Loading Amount

### 2.1. Method for Loading L-Arginine

100 mg of PCN-223-Fe particles and 200 mg of L-arginine were put in 10 ml of distilled water (DI water) and then stirred at 100 rpm for 2 days at room temperature so that a sufficient amount of L-arginine was adsorbed to the PCN-223-Fe particles.

Then, L-arginine-loaded particles (L-arg@MOF) were obtained by washing with ethanol using a filter paper having a pore size of 0.2 μm, and then dried at 60° C. for 3 hours to finally ensure particles loaded with L-arginine (L-arg@MOF).

### 2.2. Analysis of L-Arginine Loading Amount

A suspension was prepared by suspending L-arginine-loaded particles (L-arg@MOF) in distilled water to reach a concentration of 100 μg/ml. Then, the suspension was stored in a shaking incubator for 3 days to allow L-arginine to be sufficiently released from the PCN-223-Fe particles. Thereafter, the suspension was centrifuged at 13,000 rpm for 10 minutes, and the supernatant was collected, then a loading amount of L-arginine was analyzed by high-performance liquid chromatography-mass spectrometry (HPLC-MS). Assay conditions were as follows:
1) Mobile phase: mobile phase in which a ratio of distilled water containing 0.1% formic acid to acetonitrile is 3:7
2) Flow rate: 0.5 ml/min
3) m/z: 254 (positive mode)

### 2.3. Analysis of L-Arginine Release Profile (L-Arginine Release Experiment)

5 mg of L-arginine-loaded particles (L-arg@MOF) was suspended in 5 ml of PBS at pH 5.5. 1 ml of the suspension was collected at designated times (1, 2, 4, 8, 12, 24, 48 and 72 hours), followed by adding 1 ml of fresh PBS at pH 5.5 again. Then, the L-arginine release amount was analyzed by HPLC-MS under the same condition as the L-arginine loading amount analysis method.

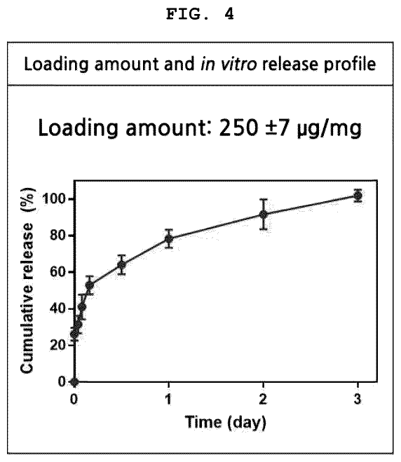

As a result of the experiment, the L-arginine loading amount was 270±7 μg/mg, and the in-vitro L-arginine release profile is shown in FIG. 4.

### 3. Anticancer Drug Loading and Loading Amount Analysis

### 3.1. Method for Loading Irinotecan (IRI)

50 mg of PCN-223-Fe particles and 50 mg of irinotecan as an anticancer drug were put in 50 ml of distilled water, and stirred at 100 rpm for 2 days at room temperature so that a sufficient amount of irinotecan was adsorbed to the PCN-223-Fe particles.

Then, irinotecan-loaded particles (IRI@MOF) were obtained by washing with ethanol using a filter paper having a pore size of 0.2 μm, and then dried at 60° C. for 3 hours to finally ensure particles loaded with irinotecan (IRI@MOF).

### 3.2. Assay of Irinotecan Loading Amount

A suspension was prepared by suspending irinotecan-loaded particles (IRI@MOF) in distilled water to reach a concentration of 100 μg/ml. Then, the suspension was stored in a shaking incubator for 3 days to allow irinotecan to be sufficiently released from the PCN-223-Fe particles. Thereafter, the suspension was centrifuged at 13,000 rpm for 10 minutes, and the supernatant was collected, then a loading amount of irinotecan was analyzed by HPLC-MS. Analysis conditions were as follows:
1) Mobile phase: mobile phase in which a ratio of 10 mM ammonium acetate to methanol is 3:7
2) Flow rate: 0.5 ml/min
3) m/z: 587 (positive mode)

### 3.3. Analysis of Irinotecan Release Profile (Irinotecan Release Experiment)

5 mg of irinotecan-loaded particles were suspended in 5 ml of PBS at pH 5.5. 1 ml of the suspension was collected at designated times (1, 2, 4, 8, 12, 24, 48 and 72 hours), followed by adding 1 ml of fresh PBS at pH 5.5 again. Then, the irinotecan release amount was analyzed by HPLC-MS under the same condition as the irinotecan loading amount analysis method.

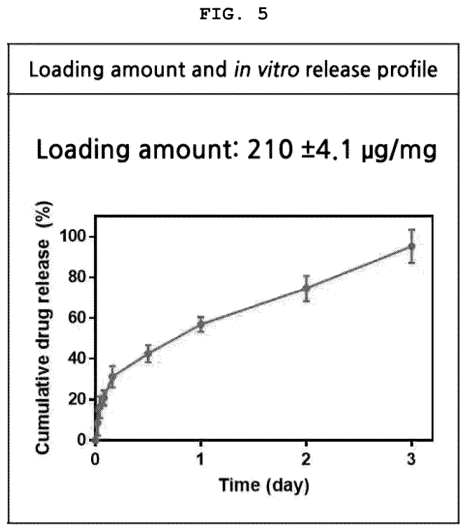

As a result of the experiment, the loading amount of irinotecan was 210±4.1 μg/mg, and the in-vitro release profile of irinotecan is shown in FIG. 5.

### 4. NO Generation Experiment

Generation of nitric oxide was investigated by reacting the above-described compounds having a structure of

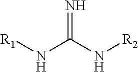

with hydrogen peroxide (H₂O₂) in the presence of a catalyst. After suspending particles including 1 mg/ml of the compound below and the catalyst in a medium composed of 5 ml of PBS at pH 5.5 and hydrogen peroxide (H₂O₂) at 1 mM concentration, the suspension was subjected to incubation at 37° C. for 4 hours, followed by sampling the supernatant to analyze the NO generation through Griess assay. Results thereof are shown in Table 1 below.

TABLE 1

| | | Concentration |
|---|---|---|
| | | of |
| Compound | Catalyst | generated NO |
| 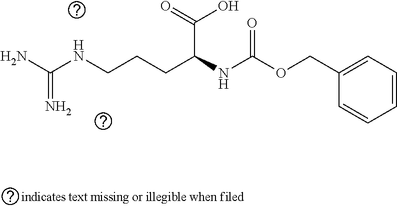 [SMILES: N=C(N)NCCC[C@H](NC(=O)OCc1ccccc1)C(=O)O] | PCN-223-Fe 1 mg/ml Mn-porphyrin 2 mg/ml | 216 μM 34 μM |
| Nα-Benzyloxycarbonyl-L- | | |
| arginine, MW: 308.33 g/mol | | |
| 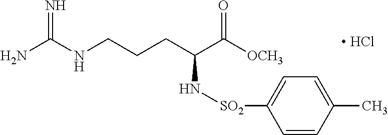 [SMILES: COC(=O)[C@H](CCCNC(=N)N)NS(=O)(=O)c1ccc(C)cc1 ; "· HCl"] | PCN-223-Fe 1 mg/ml Mn-porphyrin 2 mg/ml | 187 μM 45 μM |
| Nα-Tosyl-L-arginine Methyl | | |
| Ester Hydrochloride, MW: | | |
| 378.9 g/mol | | |
| 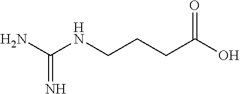 | PCN-223-Fe 1 mg/ml Mn-porphyrin 2 mg/ml | 340 μM 16 μM |
| γ-Guanidinobutyric acid, | | |
| MW: 145.16 | | |

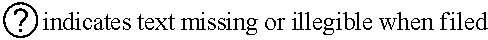

Further, 2 mg of L-arginine-loaded particles (L-arg@MOF) and 3 mg of irinotecan-loaded particles (IRI@MOF) were suspended in a medium composed of 5 ml of PBS at pH 5.5 and hydrogen peroxide, and then the supernatant was sampled at (1, 2, 4, 8, 12, 24, 48, and 72 hours) to analyze the NO generation.

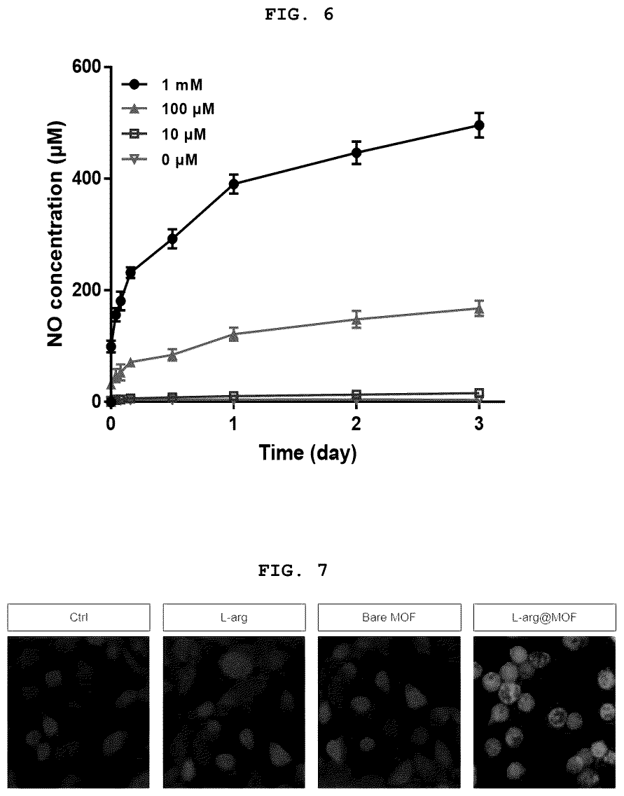

Using H₂O₂ concentrations of 0 μM, 10 μM, 100 μM, and 1 mM as experimental groups, NO generation according to the concentration of H₂O₂ was analyzed, and results thereof are shown in FIG. 6.

### 5. Analysis of Intracellular NO Generation

MCF-7/ADR, a multidrug-resistant cancer cell, was prepared at 1×10⁶ cell/well, and then cultured in DMEM (10% fetal bovine serum (FBS), 100 μg/ml streptomycin and 100 U/ml penicillin) medium for 24 hours at 37° C. with 5% CO₂.

Then, the following experimental groups were set for comparing the degree of intracellular NO generation.
1) No treatment
2) L-arginine solution treatment
3) L-arginine non-loaded MOF (Bare MOF) treatment
4) L-arginine-loaded MOF (L-arg@MOF) treatment

The samples corresponding to each group were treated, and incubated for 24 hours at 37° C. with 5% CO₂, then treated with DAFDA (3-amino, 4-aminomethyl-2′,7′difluorescein diacetate) as a NO probe at 10×10⁻⁶ M, followed by further incubating for 30 minutes at 37° C. with 5% CO₂.

Using a confocal laser scanning microscope (CLSM; TCS SP8 STED CW, Leica Microsystems, Germany), intracellular NO generation (excitation: 488 nm/emission: 515 nm) was analyzed, and results thereof are shown in FIG. 7.

### 6. Evaluation of Antitumor Efficacy at the Cellular Level

MCF-7/ADR, a multidrug-resistant cancer cell, was prepared at 5×10³ cell/well, and then cultured in DMEM (10% fetal bovine serum (FBS), 100 μg/ml streptomycin and 100 U/ml penicillin) medium for 24 hours at 37° C. with 5% CO₂.

Then, the following experimental groups were set for comparing antitumor efficacy.
1) Irinotecan solution treatment
2) Irinotecan solution+L-arg@MOF treatment
3) IRI@MOF treatment
4) IRI@MOF+L-arg@MOF treatment

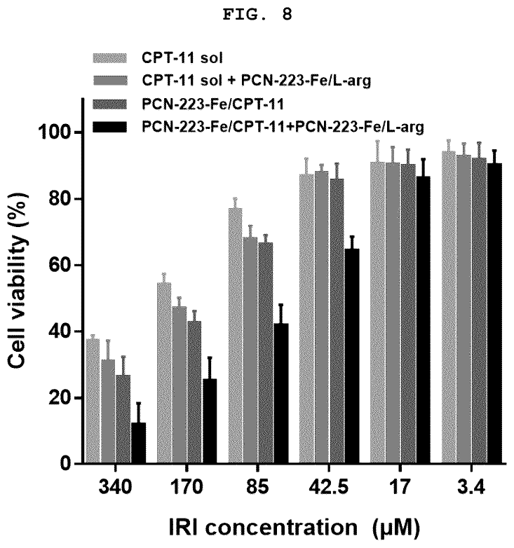

The samples corresponding to each group were treated, and further cultured for 2 days at 37° C. with 5% CO₂, then cells were measured using a kit (Ez-Cytox; Daeil Lab Service, Seoul, Korea) to measure cell viability. The survival rate was analyzed, and results thereof are shown in FIG. 8.

1-13. (canceled)

14. A method for generating nitric oxide, the method comprising:
administrating to a subject in need thereof a composition comprising:
a material containing a transition metal; and
(i) L-arginine and/or (ii) a compound represented by Formula 1 or a salt thereof: 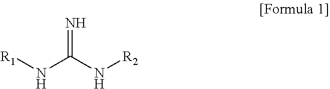
wherein R₁ is hydrogen or an alkyl group having 1 to 5 carbon atoms; and
R₂ is a substituted or unsubstituted alkyl group having 1 to 30 carbon atoms.

15. The method of claim 14, wherein the composition comprises the compound represented by Formula 1 or the salt thereof, and R₂ is the substituted alkyl group substituted with a carboxyl group or an ester group having 1 to 10 carbon atoms.

16. The method of claim 14, wherein the material is a carrier, and the composition comprises the compound supported on the carrier.

17. The method of claim 14, wherein the transition metal is one selected from the group consisting of iron, manganese, zirconium, copper, nickel, cobalt and zinc.

18. The method of claim 16, wherein the carrier is a metal-organic framework.

19. The method of claim 18, wherein the metal-organic framework includes a porphyrin ligand.

20. The method of claim 14, wherein the composition comprises the compound represented by Formula 1 or the salt thereof, and the compound is selected from the group consisting of the following compounds:

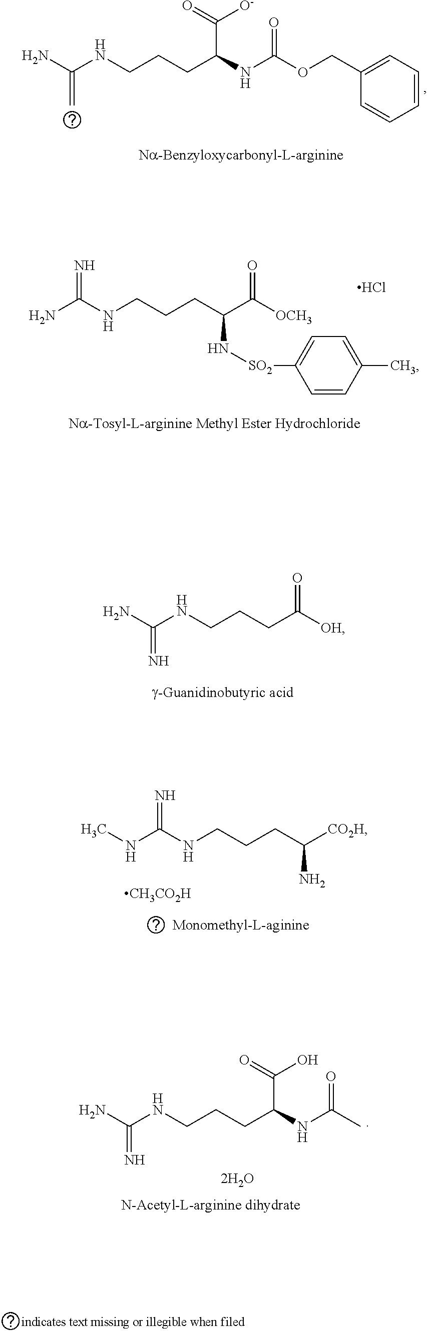

21. The method of claim 14, wherein the material is PCN-223 or Mn-porphyrin.

22. A method for treating cancer, the method comprising:
administrating to a subject in need there of a composition comprising:
a material containing a transition metal;
L-arginine and/or a compound represented by Formula 1, or a salt thereof: 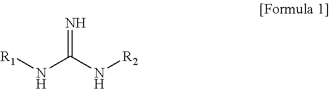
wherein R₁ is hydrogen or an alkyl group having 1 to 5 carbon atoms; and
R₂ is a substituted or unsubstituted alkyl group having 1 to 30 carbon atoms.

23. The method of claim 22, wherein the composition comprises the compound represented by Formula 1 or the salt thereof, and R₂ is the substituted alkyl group substituted with a carboxyl group or an ester group having 1 to 10 carbon atoms.

24. The method of claim 22, wherein the material is a metal-organic framework carrier including a porphyrin ligand, and the composition comprises the compound supported on the metal-organic framework carrier.

25. The method of claim 22, wherein the transition metal is one selected from the group consisting of iron, manganese, zirconium, copper, nickel, cobalt and zinc.

26. The method of claim 22, wherein the composition comprises the compound represented by Formula 1 or the salt thereof, and the compound is selected from the group consisting of the following compounds: 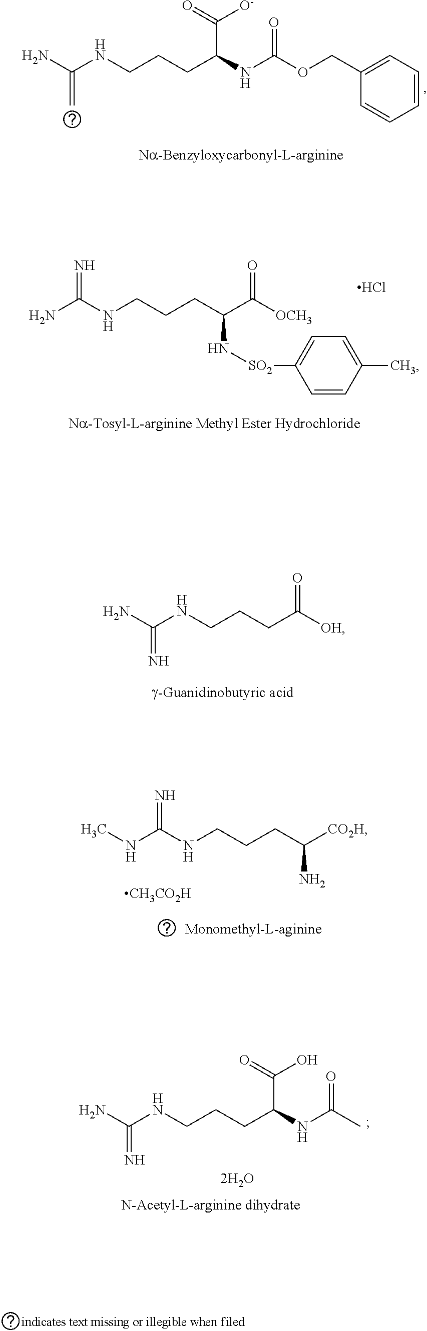
and
the material is PCN-223 or Mn-porphyrin.

27. A method for inhibiting anticancer drug resistance, the method comprising:
administrating to a subject in need there of a composition comprising:
a material containing a transition metal;
L-arginine and/or a compound represented by Formula 1, or a salt thereof: 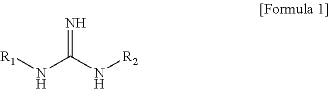
wherein R₁ is hydrogen or an alkyl group having 1 to 5 carbon atoms; and
R₂ is a substituted or unsubstituted alkyl group having 1 to 30 carbon atoms. administrating to a subject in need there of a composition comprising:
a material containing a transition metal;
L-arginine and/or a compound represented by Formula 1, or a salt thereof: 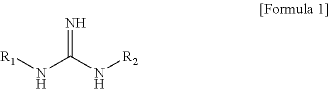
wherein R₁ is hydrogen or an alkyl group having 1 to 5 carbon atoms; and
R₂ is a substituted or unsubstituted alkyl group having 1 to 30 carbon atoms.

28. The method of claim 27, wherein the anticancer drug is Irinotecan, Eribulin, Carboplatin, Cisplatin, Halaven, 5-fluorouracil (5-FU), Gleevec, Vincristine, Vinblastine, Vinorelvine, Paclitaxel, Docetaxel, Etoposide, Topotecan, Dactinomycin, Doxorubicin, Daunorubicin, Valrubicin, Flutamide, Gemcitabine, Mitomycin, Bleomycin, Capecitabine, Tamoxifen, Belotecan, Imatinib, Streptozocin, Mechlorethamine, Chlorambucil, Doxifluridine, Etoposide, Idarubicin, Letrozole, Leucovorin, Levamisole, Teniposide, Tretinoin, Sorafenib, Mitoxantrone, Thioguanine, Pipobroman, Mesna, Nofetumomab, Oprelvekin, Cytarabine, Dacarbazine, and/or Afinitor.

29. The method of claim 27, wherein the composition comprises the compound represented by Formula 1 or the salt thereof, and R₂ is the substituted alkyl group substituted with a carboxyl group or an ester group having 1 to 10 carbon atoms.

30. The method of claim 27, wherein the material is a metal-organic framework carrier including a porphyrin ligand, and the composition comprises the compound supported on the metal-organic framework carrier.

31. The method of claim 27, wherein the transition metal is one selected from the group consisting of iron, manganese, zirconium, copper, nickel, cobalt and zinc.

32. The method of claim 27, wherein the composition comprises the compound represented by Formula 1 or the salt thereof, and the compound is selected from the group consisting of the following compounds:

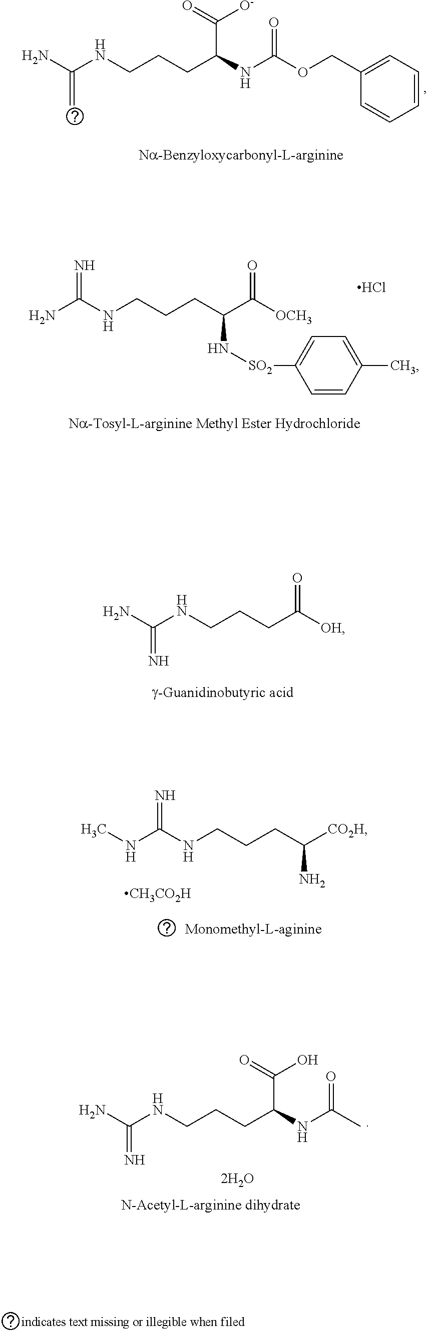

33. The method of claim 27, wherein the material is PCN-223 or Mn-porphyrin.
